SEQUENCE LISTING (16 sequences)

SEQ ID NO: 1 - DNA, 32 bp, Artificial
  Primer
aagcttccaccatggcccaggtgcagctggtg

SEQ ID NO: 2 - DNA, 34 bp, Artificial
  Primer
ggatcctgtcgacccctagaacggtgaccttggt

SEQ ID NO: 3 - DNA, 93 bp, Artificial
  Artificial epitope construct
aagcttcaccatggcccctgacaccagacctgcccctggatctaccgctcctcctgcccacggagtcacaagcgcacctccggacacaaggcc

SEQ ID NO: 4 - DNA, 93 bp, Artificial
  Artificial epitope construct
ggatcctgtcgactcgggagctgaggtgacaccatgagctgggggggctgttgagcctggggcgggccttgtgtccggaggtgcgcttgtgac

SEQ ID NO: 5 - DNA, 156 bp, Artificial
  Artificial epitope construct
aagcttcaccatggcccctgacaccagacctgcccctggatctaccgctcctcctgcccacggagtcacaagcgcacctccggacacaaggcccgccccaggctcaacagccccccagctcatggtgtcacctcagctcccgagtcgacaggatcc

SEQ ID NO: 6 - DNA, 157 bp, Artificial
  Artificial epitope construct
aagcttcaccatggcccctgacaccagacctgcccctggatctaccgctcctcctgcccacggagtcacaagcgcacctccggacacaaggcccgccccaggctcaacagcccccccagctcatggtgtcacctcagctcccgagtcgacaggatcc

SEQ ID NO: 7 - DNA, 33 bp, Artificial
  Primer
cccaggctcaacagccggcccagctcatggtgt

SEQ ID NO: 8 - DNA, 33 bp, Artificial
  Primer
acaccatgagctgggccggctgttgagcctggg

SEQ ID NO: 9 - DNA, 157 bp, Artificial
  Artificial epitope construct
aagcttcaccatggcccctgacaccagacctgcccctggatctaccgctcctcctgcccacggagtcacaagcgcacctccggacacaaggcccgccccaggctcaacagccggcccagctcatggtgtcacctcagctcccgagtcgacaggatcc

SEQ ID NO: 10 - DNA, 5464 bp, Artificial
  Engineered construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttccaccatggcccaggtgcagctggtgcagtctggagctgaggtgaagaagcctggggcctcagtgaaggtctcttgcaaggcttctggatacaccttcaccggctactatatgcactgggtgcgacaggcccctggacaagggcttgagtggatgggatggatcaaccctaacagtggtggcacaaactatgcacagaagttccagggcagagtcaccattaccagggacacatccgcgagcacagcctacatggagctgagcagcctgagatctgaagacacggctgtgtattactgtgcgagagatttttggagtggttaccttgactactggggccagggaaccctggtcaccgtctcgagaggtggaggcggttcaggcggaggtggctctggcggtggcggatcgcagtctgctctgactcagcctgcctccgtgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccatctctgggctccaggctgaggacgaggctgattattactgcagctcatatagaagcagtaacacttgggtgttcggcggagggaccaaggtcaccgttctagggtcgacggtaccgcgggcccgggatccaccggtcgccaccattgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 11 - DNA, 3621 bp, Artificial
  Engineered construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcaccatggcccctgacaccagacctgcccctggatctaccgctcctcctgcccacggagtcacaagcgcacctccggacacaaggcccgccccaggctcaacagccggcccagctcatggtgtcacctcagctcccgagtcgacggtaccgcgggcccgggatccaccggtcgccaccattgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccacc

SEQ ID NO: 12 - DNA, 5438 bp, Artificial
  Engineered construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagttggatccaccggtcgccaccattgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 13 - DNA, 6877 bp, Artificial
  Engineered construct
gcgcgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcaccatggcccctgacaccagacctgcccctggatctaccgctcctcctgcccacggagtcacaagcgcacctccggacacaaggcccgccccaggctcaacagccggcccagctcatggtgtcacctcagctcccgagtcgacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaaaggatccgaacaaaaactcatctcagaagaggatctgaatatgcataccggtcatcatcaccatcaccattgagtttgatccccgggaattcagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctcgtcgagctagcttcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctcgttggaatttgccctttttgagtttggatcttggttcattctcaagcctcagacagtggttcaaagtttttttcttccatttcaggtgtcgtgaacacgtggtcgcggccgcaagcttccaccatggcccaggtgcagctggtgcagtctggagctgaggtgaagaagcctggggcctcagtgaaggtctcttgcaaggcttctggatacaccttcaccggctactatatgcactgggtgcgacaggcccctggacaagggcttgagtggatgggatggatcaaccctaacagtggtggcacaaactatgcacagaagttccagggcagagtcaccattaccagggacacatccgcgagcacagcctacatggagctgagcagcctgagatctgaagacacggctgtgtattactgtgcgagagatttttggagtggttaccttgactactggggccagggaaccctggtcaccgtctcgagaggtggaggcggttcaggcggaggtggctctggcggtggcggatcgcagtctgctctgactcagcctgcctccgtgtccgggtctcctggacagtcagtcaccatctcctgcactggaaccagcagtgacgttggtggttataactatgtctcctggtaccaacagcacccaggcaaagcccccaaactcatgatttatgaggtcagtaagcggccctcaggggtccctgatcgcttctctggctccaagtctggcaacacggcctccctgaccatctctgggctccaggctgaggacgaggctgattattactgcagctcatatagaagcagtaacacttgggtgttcggcggagggaccaaggtcaccgttctagggtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagattcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgcca

SEQ ID NO: 14 - protein, 49 aa, Artificial
  Artificial epitope construct
MAPDTRPAPGSTAPPAHGVTSAPPDTRPAPGSTAPPAHGVTSAPESTGS

SEQ ID NO: 15 - protein, 35 aa, Artificial
  Artificial epitope construct
MAPDTRPAPGSTAPPAHGVTSAPPDTRPAPGSTAP

SEQ ID NO: 16 - protein, 49 aa, Artificial
  Artificial epitope construct
MAPDTRPAPGSTAPPAHGVTSAPPDTRPAPGSTAGPAHGVTSAPESTGS

(19) United States
(12) Patent Application Publication
(10) Pub. No.: US 2004/0115740 A1
(43) Pub. Date: Jun. 17, 2004

(54) Intracellular analysis

(76) Inventor: Roderick Simon Patrick Benson, Burnage (GB)

Correspondence Address: Martin A. Hay, 13 Queen Victoria Street, Macclesfield Cheshire UK, SK11 6LP, GB

(21) Appl. No.: 10/471,607

(22) PCT Filed: Apr. 2, 2002
(86) PCT No.: PCT/GB02/01235

(30) Foreign Application Priority Data
Mar. 31, 2001 (GB) 0108165.2

(51) Int. Cl. G01N 33/53; G01N 33/567

(52) U.S. Cl. 435/7.2

(57) ABSTRACT

The present invention relates to a method for the intracelular analysis of a target molecule, e.g. to detect the presence and/or amount thereof and to cells for use in such assays.

The present invention relates to a method for the intracellular analysis of a target molecule, e.g. to detect the presence and/or amount thereof and to cells for use in such assays.

Within the field of cell biology it is fundamentally desirable to study the presence, or otherwise, and interactions of intracellular molecules. Many techniques by which such intracellular molecules may be studied are known in the art. They include immunocytochemistry and radio-immunoassays.

A common limitation of such assays is that they require the permeabilisation or mechanical disruption of the cell membrane of the cell to be studied in order that the chosen molecule may be assessed. For instance immunocytochemistry is most frequently performed on fixed cells treated with detergent, or other such agents capable of puncturing the plasma membrane, to allow antibodies to enter the cell. Similarly it is normal to conduct radio-immunoassays on cells that have been fragmented in order that their contents are more readily accessible. The known techniques are, therefore, unsuitable for the study of intracellular molecules within living cells.

EP-A-0 969 284 concerns the use of “fluorogenic vectors” to allow marking of specific intracellular targets. Such vectors comprise a membrane translocation portion that enables the vector to cross the cell membrane, a fluorophore that reports the presence and location of the vector within the cell, and a specifying component (such as an antibody) that enables the vector to bind specifically to its target molecule. In use the vectors are administered extracellularly. The action of the membrane translocation portion enables the vector to enter a cell to be tested, wherein the specifying component causes the vector to bind to its target molecule, if present. Illumination of the cell with light of the excitation wavelength of the chosen fluorophore causes the fluorophore to emit light at its emission wavelength. This emitted light enables the vector to be visualised within the cell. In certain cases the interaction between intracellular components may be studied by targeting the components with vectors having fluorophores that are able to act as fluorescence resonance energy transfer (FRET) partners. The use of the vectors allows dynamic studies of the localisation and interactions of cellular molecules within the cell.

The disclosure of EP-A-0 969 284 suffers from a number of important limitations, of which the greatest is that it is not possible to differentiate between vectors that have bound to their target and those that remain unbound within the cell. As such the vectors described have only limited utility.

WO-A-9840477 discloses fluorescent protein sensors for detection of analytes. The sensors are expressed intracellularly and have a binding protein moiety, a donor fluorescent protein moiety and an acceptor fluorescent protein moiety. The binding protein moiety has an analyte binding region, to which an analyte binds, causing the indicator to change conformation in the presence of the analyte. Upon binding of the analyte to the analyte binding region the donor and acceptor fluorescent protein moieties change their positions relative to each other. The donor and acceptor fluorescent moieties are able to act as FRET partners when the donor moiety is excited and the distance between the donor moiety and the acceptor moiety is small. These indicators can be used to measure analyte concentrations in samples, such as calcium ion concentrations in cells. The specific embodiment of WO-A-9840477 utilises intracellularly expressed constructs which encode Ca²⁺ binding protein moieties and complementary target protein moieties. These binding protein and target protein moieties are respectively exemplified by calmodulin and the M13 calmodulin-binding region of calmodulin-dependent kinase. The binding and target protein moieties are coupled to fluorophores able to act as FRET partners. In the presence of Ca²⁺ the affinity of the binding protein for its complementary target protein is increased causing the binding protein and target to interact. This in turn brings about an increased proximity between the fluorophores, thereby enabling FRET to occur between them when suitably excited. Thus the presence of Ca²⁺ is indicated by a change in the fluorescence emission spectrum. A similar technique, in which cAMP is the analyte and protein kinase A the binding protein, is disclosed in Zaccolo et al., 2000.

According to a first aspect of the invention there is provided a method of intracellularly analysing for a target molecule within a biological cell, the method comprising the steps of

i) expressing within the cell a first polypeptide sequence comprised of a first binding species capable of binding to the target molecule and a first reporter moiety attached to the first binding species;

ii) expressing within the cell a second polypeptide sequence comprised of a second binding species capable of competing with the target molecule for binding of the first binding species and a second reporter moiety, said first and second reporter moieties being such that on binding together of the first and second binding species the first and second reporter moieties interact so as to be capable of producing a signal that can be differentiated from one capable of being generated when said first and second reporter moieties do not interact; and

iii) effecting a measurement to determine the presence or otherwise of a signal representative of binding of the first and second binding species.

The first and second polypeptide sequences may be expressed as separate molecules within the biological cell to be analysed. Alternatively a single molecule which contains both the first and second polypeptide sequences may be expressed. In the case where both polypeptide sequences are contained within a single molecule the two sequences may be directly linked with one another, or alternatively they may be separated by a number of amino acid residues that do not form part of either sequence.

The method of the invention allows intracellular analysis for a target molecule (e.g. to determine the presence (or otherwise) and/or amount thereof) within a cell without the need for rupturing of the cell membrane to introduce the investigating species into the cell.

More particularly, a cell to be assayed by the method of the invention may be transfected (using techniques well known in the art) with DNA sequences capable of being expressed within the cell to generate (i) a first polypeptide sequence incorporating a (first) binding species and a (first) reporter moiety, and (ii) a second polypeptide sequence incorporating a (second) binding species and (second) reporter moiety. It will be appreciated that the first and second binding species and reporter moieties are all polypeptides.

The first binding species is capable of complex formation with the target molecule of interest. The second binding species is capable of complex formation with the first binding species and as such is capable of competing with the target molecule for complex formation with the first binding species.

Each of the first and second binding species has a respective reporter moiety attached thereto. These reporter moieties, and their attachment to the respective binding species, are such that when a complex is formed between said first and second binding species the reporter moieties interact so as to be capable of generating a signal different from that generated when there is no such binding.

In the absence of the target molecules within the cell, the first and second binding species complex with each other so that the interaction of the reporter moieties enables a signal to be generated that is indicative of such binding, thereby demonstrating that the target molecule is not present in the cell.

In the theoretical situation that the target molecule is present in the cell in an amount such that the first binding species is exclusively bound thereto (i.e. there is no complex formation between the first and second binding species) the first and second reporter moieties do not interact. The aforementioned signal cannot therefore be generated, thus indicating that the target molecule is present in the cell.

It will be appreciated that; in practice, conditions within the cell will often lie between the two extremes outlined above. In these conditions the amount, or otherwise, of the target within the cell will be reflected in the ratio of the signal indicative of binding and the signal indicating a lack thereof.

The method of the invention may also be used for the quantitative determination of the amount of target molecule present in a cell as indicated by the ratio of the intensity of the signal indicative of binding to the intensity of the signal indicating a lack thereof.

It will be appreciated that the first binding species should not bind a region of the target molecule that is critical for its function. Thus, for example, when the target molecule is a cell cyclin the first binding species may disable the cyclin's normal function. This may result in the expression level of the cyclin itself being altered as a consequence of the binding of the antibody causing the cell cycle to arrest.

It will also be appreciated that if the target molecule is found in a particular compartment of the cell then it is preferable to ensure the presence of the first and second polypeptide sequences within that compartment This is preferably achieved by targeting said first and second polypeptide sequences to the chosen compartment. Suitable methods by which such targeting may be achieved include the provision on the polypeptide sequences of “targeting sequences”, an example of which is the KDEL amino acid quartet which causes retention in the endoplasmic reticulum. Other such targeting sequences conferring different specificities are well known to those skilled in the art. If desired the invention may alternatively be put into practice by expression of the first and second polypeptide sequences at the site of the intracellular compartment of interest.

It is particularly preferred that the reporter moieties are each fluorescent proteins having substantially overlapping absorption/emission spectra such that, when the two fluorescent proteins are in sufficiently close proximity, one of the fluorophores (when excited) acts as a donor and is capable of effecting Fluorescent Resonance Energy Transfer to the other fluorophore which acts as an acceptor (for a more detailed description of FRET see infra). In the context of the present invention, the two fluorophores may be brought into sufficiently close proximity upon complex formation between the first and second binding species. If no such complex formation occurs then excitation of the donor will provide an emission spectrum characteristic of that fluorophore. If however binding has occurred then excitation of the donor will result in emission characteristic of the acceptor (due to its excitation by FRET) even though the frequency of the excitation radiation is not appropriate for direct acceptor fluorescence emission.

The fluorescent protein that provides the first reporter moiety may for example be Cyan Fluorescent Protein (CFP) whereas the second reporter moiety may be provided by Yellow Fluorescent Protein (YFP). The excitation wavelength of CFP is 433 nm and its fluorescent emission is at 476 nm. The fluorescent emission of YFP is at 527 nm. If the target molecule is not present in the cell, irradiation (of the cell) with light of 433 nm will result in fluorescent emission at 527 nm (yellow); if the target molecule is present then emission at 476 nm (cyan) will be detected. If target molecule is present in the cell then the ratio of intensities of the relative emissions at 527 nm and 476 nm indicate the amount of target present.

The amino acid sequence of, and DNA encoding, YFP are identified as sequences 3 and 4 in PCT application WO-A-9806737. The amino acid substitutions by which CFP differs from GFP are listed in WO-A-9840477, wherein CFP is identified as W7.

Other combination of fluorescent proteins that may be used include, Blue Fluorescent Protein (BFP) and Green Fluorescent Protein (GFP). The DNA and amino acid sequences encoding GFP are identified as sequences 1 and 2 in WO-A-9806737. The amino acid substitutions by which BFP differs from GFP are disclosed in WO-A-9840477, in which BFP is identified as P4-3.

It is preferred that the first binding species is an antibody or fragment thereof, e.g. an intrabody, all of which are for convenience herein embraced by the term antibody unless the context otherwise requires. The use of an antibody gives rise to various possibilities for the nature of the target molecule to be investigated and the nature of the second binding species.

In a particularly preferred first embodiment of the invention, the target molecule is a peptide antigen (and will be capable of binding to the antibody that provides the first binding species). In this case, the second binding species may also be an antigen (again capable of combining with the antibody that provides the first binding species). Most preferably the second binding species is the epitope to which the first binding species (antibody) has been raised, although we do not preclude the use of suitable antigens other than the original epitope.

It will be appreciated that for this first embodiment of the invention, the cell to be investigated is transfected with:

(a) a first nucleic acid construct that is capable of being expressed within the cell to produce the antibody having the first reporter moiety (preferably a fluorescent protein) attached thereto; and

(b) a second nucleic acid construct that is capable of being expressed within the cell to produce the peptide epitope (of the second binding species) having the second reporter moiety (preferably a fluorescent protein) attached thereto.

According to a second embodiment of the invention, the target molecule is a non-peptide antigen capable of binding to the first antibody that provides the first binding species. In this case, the second binding species may be an anti-idiotype antibody (referred to more simply as an anti-antibody) capable of binding to the binding site of the first antibody which binds the non-peptide epitope.

For this second embodiment of the invention, it will be appreciated that the cell under investigation is transfected with a nucleic acid construct of the type described for (a) above and a second nucleic acid construct similar to (b) above but capable of expressing the anti-antibody rather than the peptide antigen.

Antibodies to be used in accordance with the method of the invention may be obtained by phage display techniques that enable large numbers of recombinantly produced antibodies, or antibody fragments, to be rapidly screened for reactivity with a selected antigen. The DNA sequence encoding the selected antibody (or antibodies) may then be readily sequenced allowing its incorporation into nucleic acid constructs of the invention. A review of phage display techniques may be found in Griffiths and Duncan 1998.

For all embodiments of the invention, the nucleic acid constructs (with which the cell is transfected) for expressing the first and second polypeptide sequences may be introduced into the cell using vectors which are well known in the art. It is possible for the nucleic acid construct for expressing the first polypeptide sequence to be incorporated in a separate vector from that of the construct expressing the second polypeptide sequence, each such construct being under the control of a respective promoter. It is also possible for the two constructs to be incorporated in the same vector but to be under the control of separate promoters within the vector. An example of such a vector is pBudCE4.1 (Invitrogen, Paisley, UK).

It is most preferred that both the first and second sequences are translated from the same DNA sequence, thus producing a single mRNA transcript. This achieved by having the DNA constructs which encode the first and second polypeptide sequences under the control of a single promoter. This creates a 1 to 1 expression ratio that will maximise the sensitivity of the method of the invention. In order that the first and second polypeptide sequences may be translated from a single mRNA transcript it is preferred that the mRNA contain an internal ribosome entry sequence (IRES). A corresponding DNA sequence encoding such an IRES may therefore be incorporated in the DNA construct that encodes the two polypeptide sequences. Examples of such IRESes are well known to those skilled in the art, and include such commercially available IRESes as pIRES, produced by Clontech.

In this second embodiment of the invention it may be preferred that the first and second polypeptide sequences be linked by a chain of amino acids. Such an arrangement has the advantage that the first and second binding species (and hence their attached reporter moieties) cannot become located in separate intracellular compartments, thereby ensuring that they are able to interact as FRET partners. A linker chain suitable for use in the invention may comprise between about one amino acid residue and about thirty amino acid residues. An example of such a linker chain may consist of glycine residues linked together (-GlyGly-). In the case that the first and second reporter moieties are fluorophores that act as FRET partners, the linker chain should have a length such that the FRET partners are able to achieve a separation greater than three times the relevant Förster distance.

Whilst the invention has so far been described with reference to a method of intracellular analysis, it will be appreciated that according to a second aspect the invention provides a biological cell transfected with:

i) a first nucleic acid sequence encoding a first polypeptide sequence comprised of a first binding species capable of binding to a putative target molecule in the cell and a first reporter moiety attached to the first binding species;

ii) a second nucleic acid sequence encoding a second polypeptide sequence comprised of a second binding species capable of competing with the target molecule for binding of the first binding species and a second reporter moiety, said first and second reporter moieties being such that on binding together of the first and second binding species the first and second reporter moieties interact so as to be capable of producing a signal that can be differentiated from one capable of being generated when said first and second reporter moieties do not interact.

The invention further provides a method of producing cells as described in the preceding paragraphs the method comprising transfecting a biological cell with:

i) a first nucleic acid sequence encoding a first polypeptide sequence comprised of a first binding species capable of binding to a putative target molecule in the cell and a first reporter moiety attached to the first binding species;

ii) a second nucleic acid sequence encoding a second polypeptide sequence comprised of a second binding species capable of competing with the target molecule for binding of the first binding species and a second reporter moiety, said first and second reporter moieties being such that on binding together of the first and second binding species the first and second reporter moieties interact so as to be capable of producing a signal that can be differentiated from one capable of being generated when said first and second reporter moieties do not interact.

It is contemplated that non-human animals may be produced which contain polypeptide sequences according to any previously described aspect of the invention. Such non-human animals may include domestic animals, such as dogs, and agricultural animals such as cows, pigs or sheep. Such non-human animals may further include other vertebrates such as rodents, primates other than humans, reptiles or amphibians. Suitable non-human species may preferably include rodents such as rats, rabbits or mice.

It is further contemplated that transgenic non-human animals may be produced, containing exogenous genetic material encoding polypeptide sequences of the invention Such transgenic animals may be produced by a range of methods known to those skilled in the art, suitable methods including, but not limited to, micro-injection of genetic material, retroviral transfection, and embryonic stem cell based methods.

Production of such animals would allow studies on primary cultures taken from such animals, or even on the whole animals themselves, to be undertaken. This would provide an advantage in research science, as cell models are not always reliable in producing data which is consistent with unaltered cells in their natural state. Furthermore, studies on whole animals could be used to visualise in what organs a drug was signalling. Such studies may have utility in testing of chemotherapeutic agents, providing more information on the efficacy and toxicity of drugs being tested. Likewise polypeptide sequences according to the invention suitable for the analysis of a signalling intermediate (say the phosphorylated form of MAPK), would allow experimental procedures to be carried out, on the whole animal or derived primary cell cultures, analysis of the results from which would provide information as to how the particular treatment is interacting with the MAP pathway, and to what extent the signal is occurring in different tissues within the animal. Similarly, use of polypeptide sequences according to the invention in immune cells may help elucidate the timing and complex signalling that occurs when the immune system responds to an antigen. Finally, polypeptide sequences according to the invention, if expressed during development, may shed light on important developmental signals and their timing by indicating potential signalling pathways that may be operational during a particular period of foetal development. Other potential uses of the invention will be apparent to those skilled in the art.

The invention will now be described by way of example only with reference to the accompanying drawing, in which:

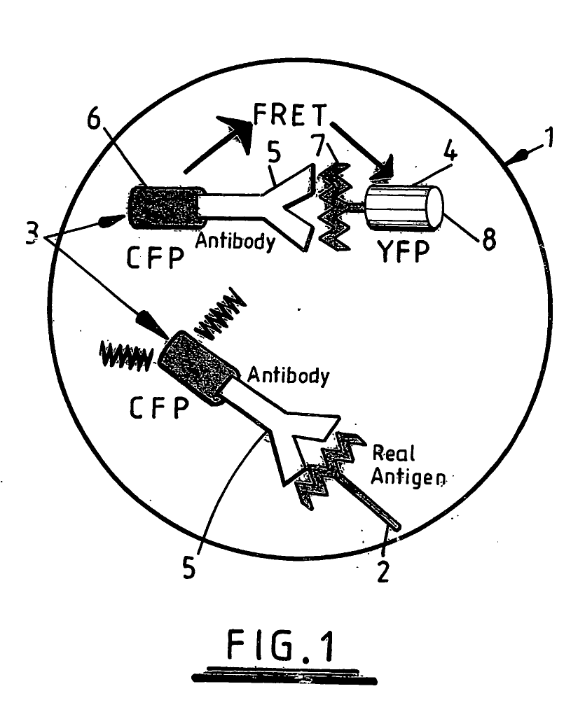

FIG. 1 schematically illustrates the method of the invention;

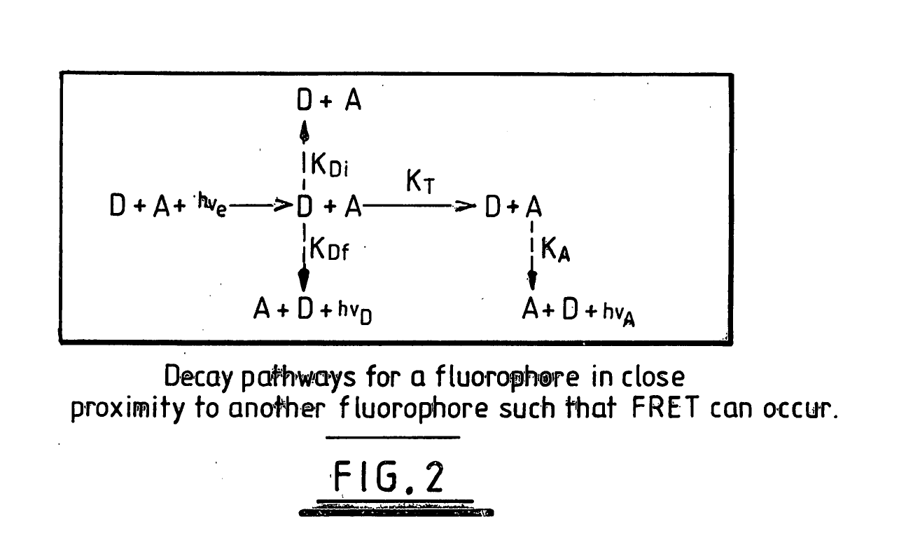

FIG. 2 illustrates decay pathways for a fluorophore in close proximity to another fluorophore such that FRET can occur;

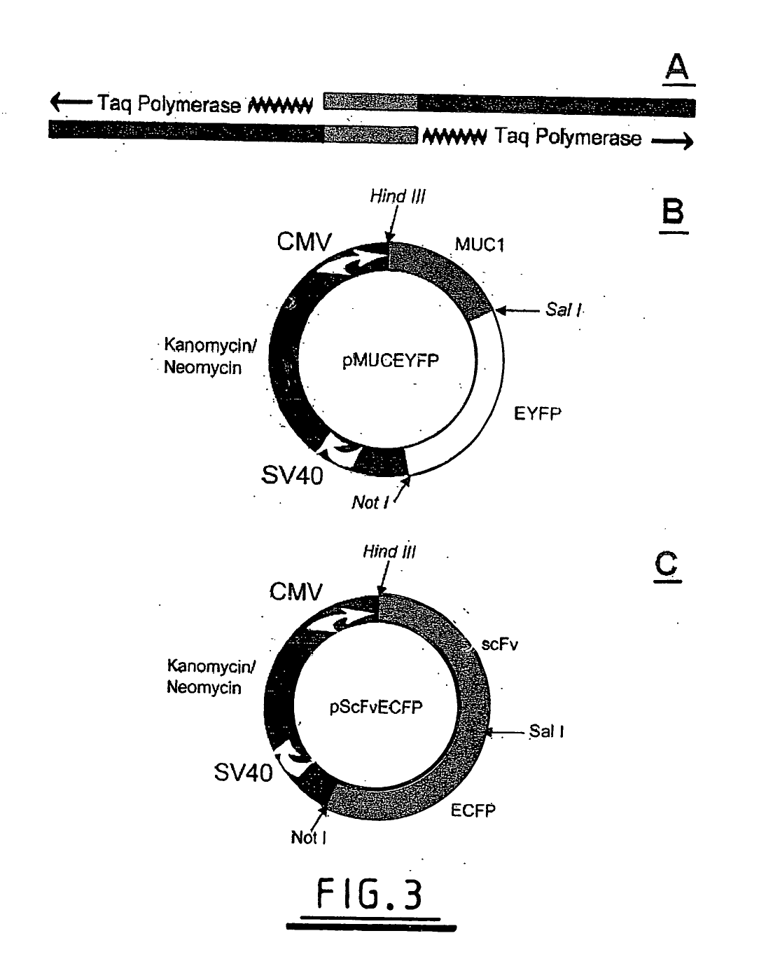

FIG. 3A shows the procedure employed in the Example (see infra) for producing a DNA sequence (the “MUC1 insert”) capable of encoding tandem repeats of the MUC1 epitope (as part of the second polypeptide);

FIG. 3B shows a vector (designated as pMUC-EYFP) incorporating the MUC1 insert and capable of expressing MUC1 coupled to YFP as the second polypeptide;

FIG. 3C shows a vector (designated pScFv-ECFP) capable of expressing an anti-MUC1 intrabody coupled to CFP as the first polypeptide;

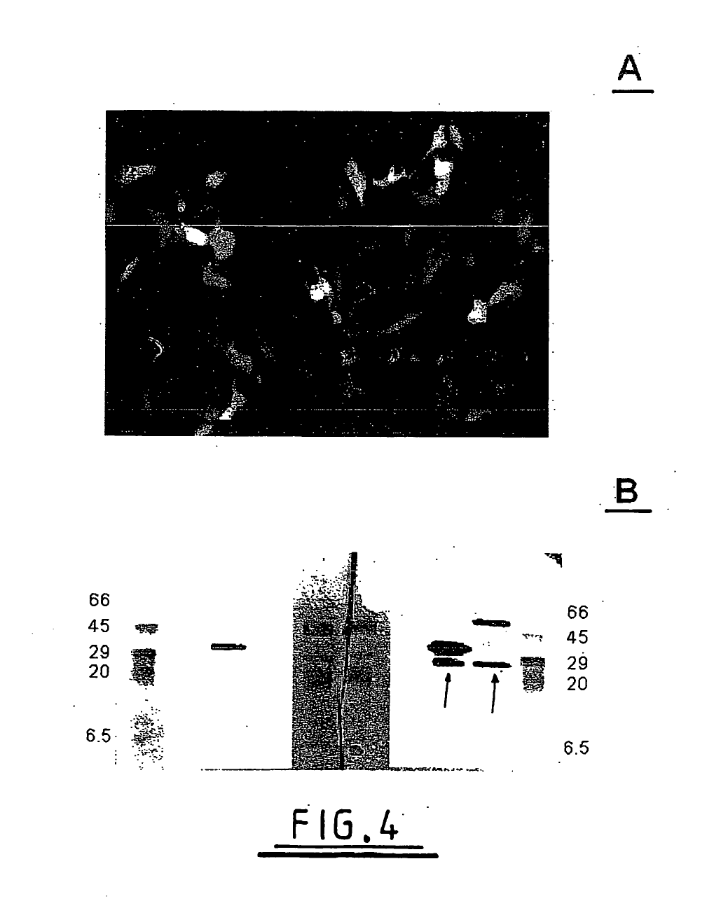

FIG. 4A shows a fluorescent microscopy image of cells expressing a second polypeptide according to the invention obtained using the procedure of the Example;

FIG. 4B shows the results of Western blotting analysis of cell lysates obtained using the procedure of the Example;

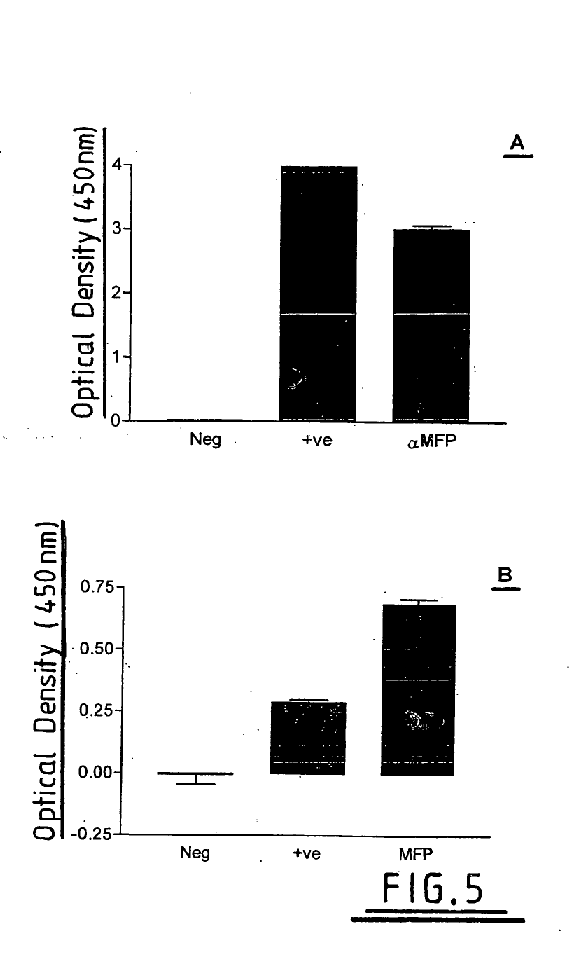

FIG. 5 shows the results of ELISA analysis of cell lysates obtained using the procedure of the Example; and

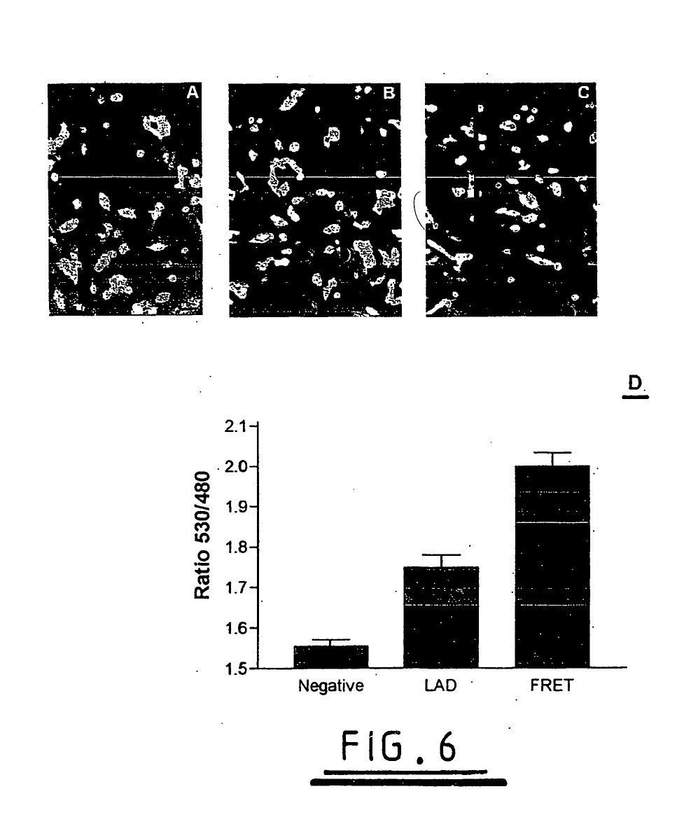

FIG. 6 shows pseudo-coloured images of cells expressing polypeptides according to the invention and control polypeptides obtained using the procedure of the Example, and quantification of fluorescent emissions by said cells.

FIG. 1 illustrates a cell 1 which endogenously produces an antigen 2 which is to be investigated by the method of the invention. Expressed within the cell (by exogenously introduced nucleic acid constructs—not shown) are first and second polypeptides 3 and 4 respectively. The first polypeptide 3 comprises an intrabody 5 attached to Cyan Fluorescent Protein (CFP) 6. The intrabody 5 is capable of binding to, and forming a complex with, the endogenous antigen 2.

The second polypeptide comprises an epitope 7 having Yellow Fluorescent Protein (YFP) 8 attached thereto.

The cell 1 is shown with two of the intrabodies 5, one bound to the artificially expressed YFP-tagged epitope 7 and the other bound to the epitope of the native cellular antigen 2.

On irradiation of the cell with 433 nm light, intrabody 5 that is bound to native cellular epitope 2 fluoresces at 476 nm (cyan). However intrabody 5 that is bound to YFP-tagged epitope transfers the excitation energy by FRET to YFP resulting in 527 nm (yellow) fluorescence.

The spatial separation of the CFP 6 and YFP 8 required for FRET to occur is described below with reference to FIG. 2. However reference is firstly made to the possibility of obtaining quantitative information from the “system” depicted in FIG. 1 by analogy with conventional radioimmunoassay.

In a radioimmunoassay the antigen is titrated against a constant amount of the same antigen which has been labelled with a radioactive isotope such as ¹²⁵I. The two populations of an antigen (unlabelled and labelled) compete for binding to a fixed concentration of antibody. Increasing amounts of unlabelled antigen result in less free antibody to bind the labelled antigen. Thus, if the amount of labelled antigen bound to antibody (calculated by measuring the radioactivity of the antibody/antigen complex after separation from unbound antigen) is measured then this amount will decrease with increasing amounts of unlabelled antigen. If different known amounts of unlabelled antigen are incubated with the antibody/labelled antigen mix, then a standard curve can be constructed by fitting the decrease in radioactivity to a one site competition sigmoidal curve. In the same way an in vitro system analogous to that shown in FIG. 1 should behave in an identical fashion; the present invention's equivalent of changing radioactivity being a change in the cyan/yellow fluorescence ratio as the level of FRET decreases with increasing concentrations of antigen which is not attached to YFP.

Reference is now made to FIG. 2 which shows decayed pathways when a fluorophore donor (D) and a fluorophore acceptor (A) are in close proximity such that FRET can occur.

In general terms, the quantum yield (Q) from a fluorophore is defined as the ratio of emitted to absorbed photons and is given by:

$$Q = \frac{{K}_{f}}{{K}_{f} + {K}_{i}} \qquad ( 1 )$$

Where K_{f} and Kᵢ are the radiative and non-radiative rate constants for depopulation of the excited state and represent the average frequency with which these stochastic processes occur. Obviously as Kᵢ increases so the quantum yield, and hence fluorescence, decreases. One potential non-radiative path for the relaxation of a fluorophore is the transfer of energy to a second fluorogenic group. Such a scheme is shown in FIG. 2. The transfer of energy from one to the other fluorophore means that the fluorophore that is losing energy by the non-radiative pathway (called the donor) will appear less fluorescent when it is in proximity to the fluorophore receiving this energy (called the acceptor). Conversely, the fluorophore that is receiving donor energy will emit photons even though the frequency of light exciting it is not at the right wavelength for direct acceptor fluorescence emission. These emitted photons will have wavelengths characteristic of the acceptor emission spectrum. Hence the process of FRET leads to a shift from donor to acceptor emission spectra when the fluorophores are excited by light which would normally give a donor emission spectrum (Van Der Meer et al., 1994). It is also possible for a fluorophore to transmit energy via a non-radiative path to a molecule which itself is not fluorescent (a “quencher”). In this situation a decrease in donor fluorophore fluorescence will be observed without any increase in fluorescence at a different wavelength.

From the scheme presented in FIG. 2 and using equation 1 it can be shown that the quantum yield in the absence of FRET is

$${Q}_{D} = \frac{{K}_{Df}}{{K}_{Df} + {K}_{Di}} \qquad ( 2 )$$

When FRET is present a further term is added so that:

$${Q}_{DA} = \frac{{K}_{Df}}{{K}_{T} + {K}_{Df} + {K}_{Di}} \qquad ( 3 )$$

Where Q_{DA} is the quantum yield in the presence of FRET and Q_{D} is the quantum yield in the absence of FRET respectively. The efficiency of FRET transfer (E) is defined as:

$$E = 1 - \frac{{Q}_{DA}}{{Q}_{D}} \qquad ( 4 )$$

Substituting equations 2 and 3 into 4 we get:

$$E = \frac{{K}_{T}}{{K}_{T} + {K}_{Df} + {K}_{Di}} \qquad ( 5 )$$

From this equation it can be seen that as K_{T} increases so the efficiency of FRET transfer approaches 1 and the quantum yield of the donor fluorescence approaches 0 (equation 3).

In 1948 Förster showed that K_{T} was related to the distance that the donor and acceptor fluorophores were from one another by the equation:

$${K}_{T} = ( {K}_{Df} + {K}_{Di} ) {( \frac{{R}_{0}}{R} )}^{6} \qquad ( 6 )$$

Where R₀ is the Förster distance which is defined as the distance between the two fluorophores where the amount of energy transferred from the donor to the acceptor fluorophore equals the amount of energy lost by the donor from all other processes including the emission of donor light fluorescence. From the scheme presented in FIG. 1 this condition is met when: K_{T}=K_{Df}+K_{Di}. We can now rewrite equation 4 in terms of distance so that:

$$E = \frac{{R}_{0}^{6}}{{R}_{0}^{6} + {R}^{6}} \qquad ( 7 )$$

The Förster distance (R₀) has been shown to be equal to:

$${R}_{0} = \sqrt[6]{\frac{9000 ( L n10 ) {κ}^{2} {Q}_{D} J}{128 {π}^{ 5} {n}^{ 4} {N}_{AV}}} \qquad ( 8 )$$

Where κ is an molecular orientation function (which can vary from 0 to 4), J is a number which represents the amount of overlap between the donor emission and acceptor excitation spectra, n is the refractive index of the medium, N_{AV} is Avogadro's number (6.023×1023) and Q_{D} is the quantum yield of the donor as described previously. Although the actual functions controlling κ are complex, in general a value of ⅔ is assumed as this is correct for fluorophores that can freely rotate. Even if this assumption does not hold, the error introduced in Rₒ grows slowly with respect to an increasing error in κ since:

R₀α³\sqrt{κ} (9)

The above theory demonstrates that the only requirements for FRET between two fluorophores are those which are present in the variables of equation 7. Furthermore, the Förster distance in equation 7 is only determined effectively by the quantum yield of the donor and the spectral overlap of the donor and acceptor fluorophores. This is advantageous because it means that the fluorophores do not have to be chemically modified to change their fluorescence but rather only have to be within the Förster distance. Thus, if an intrabody is labelled with a fluorophore and its antigen is labelled with a second fluorophore, such that the excitation and emission spectras of each fluorophore matches the conditions needed for FRET (they extensively overlap), then binding of the intrabody to its antigen will result in FRET from the fluorophore attached to the intrabody to the fluorophore attached to the antigen. Likewise, intrabody that has not bound antigen will display no FRET because FRET decreases rapidly (inversely proportional to R⁶) with the increase in molecular distance between the unbound intrabody/antigen pair. EXAMPLE

This Example illustrates an intracellular assay for the MUC1 epitope of human mucin 1.

In this Example the first polypeptide comprises an intrabody (ScFv) reactive to the MUC1 epitope attached to the fluorescent protein cyan fluorescent protein (CFP). The combination of intrabody and fluorescent protein is the expression product of a vector herein referred to as pScFv-ECFP.

The second polypeptide comprises the MUC1 epitope attached to yellow fluorescent protein (YFP). This combination of epitope and fluorescent protein is the expression product of a vector referred to as pMUC-EYFP.

Construction of pScFv-ECFP.

A pHEN1 bacterial expression vector encoding an ScFv specifically reactive with MUC1 was a gift from Dr M. J Embleton (The Paterson Institute, University of Manchester, UK) The sequence encoding the anti-MUC1 ScFv was amplified by polymerase chain reaction (PCR) with the following primers:

(Sequence ID No.1)

AAGCTTCCACCATGGCCCAGGTGCAGCTGGTG

(Sequence ID No.2)

GGATCCTGTCGACCCCTAGAACGGTGACCTTGGT

The chosen primers ensure that the amplification product of the reaction contains a Hind III and a Sal I restriction site at the 5′ and 3′ end respectively. In order to simplify sequencing and further cloning, the PCR product was first placed into pCR4-TOPO (Invitrogen, Paisley, UK) using the standard protocol supplied by Invitrogen. Sequencing was performed using a standard kit (Applied Biosystems, Warrington, UK) and sequencing primers T3 and T7 which bind either side of the PCR insert

After sequencing the DNA encoding the ScFv (the ScFv insert) was then excised from the PCR4-TOPO using Hind III and Sal I restriction enzymes and standard reaction conditions (Roche Lewes, East Sussex, UK). The ScFv insert was then ligated into the Hind III/Sal I cloning sites of a ECFP-N1 vector (Clontech, Cowley, Oxford, UK) which is commercially available for the production of Cyan Fluorescent Protein (CFP). T4 ligase 0.2 units in 10 μl (Roche Lewes, East Sussex, UK) and standard reaction conditions (16° C. for 18 hours) were used for all ligations described in this document. The result of this ligation was the production of a vector (pScFv-ECFP) shown schematically in Panel C of FIG. 3. The expression product of pDScFv-ECFP is an anti-MUC1 ScFv having a CFP molecule attached to its C-terminus (the first polypeptide).

Construction of pMUC-EYFP.

The MUC1 epitope comprises the amino acid sequence R-P-A-P-G-S-T. A 157 base pair nucleotide insert encoding the MUC1 epitope and its surrounding amino acids in a tandem repeat was created by synthesising two 93 base oligonucleotides with the following sequences:

| | |
|---|---|
| 5′AAGCTTCACCATGGCCCCTGACACCAGACCTGCCCCTGGATCTACCGCT | (Sequence ID No.3) |
| CCTCCTGCCCACGGAGTCACAAGCGCACCTCCGGACACAAGGCG3′ | |
| 5′GGATCCTGTCGACTCGGGAGCTGAGGTGACACCATGAGCTGGGGGGGCT | (Sequence ID No.4) |
| GTTGAGCCTGGGGCGGGCCTTGTGTCCGGAGGTGCGCTTGTGAC3′ | |

The last 29 bases at the 3′ end of each sequence (shown in bold above) are complementary to one another. The oligonucleotides were mixed in vitro, and the mixture heated to 96° C. The temperature of the mixture was then lowered to 55° C., a temperature at which the two oligonucleotides anneal through their complementary regions. The mixture was then heated to 72° C. and Taq polymerase (Roche, Lewes, East Sussex, UK) used to synthesise 3′ portions complementary to the remaining template DNA, thereby producing a 157 base pair double-stranded DNA molecule (FIG. 3A). The resultant coding sequence along with its amino acid translation is shown below:

| | |
|---|---|
| Hind III M A P D T R P A P G S | (Sequence ID No.5) |
| A\|AGCTTCAC\|CATG GCC CCT GAC ACC AGA CCT GCC CCT GGA TCT | |
| Nco I | |
| T A P P A H G V T S A P P D | |
| ACC GCT CCT CCT GCC CAC GGA GTC ACA AGC GCA CCT CCG GAC | |
| T R P A P G S T A P P A H G | |
| ACA AGG CCC GCC CCA GGC TCA ACA GCC CCC CCA GCT CAT GGT | |
| V T S A P E S T G S | |
| GTC ACC TCA GCT CCC GAG\|TCG ACA G\|GA TCC | |
| Sal I BamHI | |

As with the scFv insert, the MUC1 epitope construct was placed into pCR4-TOPO for sequencing. Sequence analysis showed that none of the clones obtained contained the desired sequence. The closest sequence to the desired sequence (Sequence ID No. 5) was:

| | |
|---|---|
| Hind III M A P D T R P A P G S | (Sequence ID No.6) |
| A\|AGCTTCAC\|CATG GCC CCT GAC ACC AGA CCT GCC CCT GGA TCT | |
| Nco I | |
| T A P P A H G V T S A P P D | |
| ACC GCT CCT CCT GCC CAC GGA GTC ACA AGC GCA CCT CCG GAC | |
| T R P A P G S T A P * * * * | |
| ACA AGG CCC GCC CCA GGC TCA ACA GCC CCC C A GCT CAT GGT | |
| * * * * * * * * * * | |
| GTC ACC TCA GCT CCC GAG\|TCG ACA G\|GA TCC | |
| Sal I BamH I | |

The sequence reproduced above is missing a C nucleotide at position 116 (the two nucleotides either side of the deletion are shown in bold). This vector was called pCR4MUC-DelC. The insert from this vector contains a frame shift mutation and will mean that no functional fluorescent protein will be produced as it is down stream from the missing nucleotide. In order to correct this mutagensis of pMUC-DelC was carried out using the QuikChange mutagensis kit (Strategene, Limerick, Northern Ireland) and the following mutagensis primers

(Sequence ID No.7)

CCCAGGCTCAACAGCCGGCCCAGCTCATGGTGT

(Sequence ID No.8)

ACACCATGAGCTGGGCCGGCTGTTGAGCCTGGG

Using the standard reactions conditions as prescribed by Strategene, pCRMUC-DelC was changed to

| | |
|---|---|
| Hind III M A P D T R P A P G S | (Sequence ID No.9) |
| A\|AGCTTCAC\|CATG GCC CCT GAC ACC AGA CCT GCC CCT GGA TCT | |
| Nco I | |
| T A P P A H G V T S A P P D | |
| ACC GCT CCT CCT GCC CAC GGA GTC ACA AGC GCA CCT CCG GAC | |
| T R P A P G S T A G P A H G | |
| ACA AGG CCC GCC CCA GGC TCA ACA GCC GGC CCA GCT CAT GGT | |
| V T S A P E S T G S | |
| GTC ACC TCA GCT CCC GAG\|TCG ACA G\|GA TCC | |
| Sal I BamHI | |

Sequencing of this vector confirmed that DNA encoding the desired amino acid sequence (MUC1 epitope sequence) had been successfully cloned. The DNA encoding the MUC1 epitope (MUC1 insert) was then excised using Hind III and Sal I restriction enzymes and standard reaction conditions (Roche, Lewes, East Sussex, UK) and ligated into the Hind III/Sal I cloning site of a EYFP-N1 expression vector from Clontech, which is commercially available for the production of Yellow Fluorescent Protein (YFP) The resultant vector was named pMUC-EYFP the structure of which is shown schematically in Panel B of FIG. 3. Expression of pMUC-EYFP produced a molecule with two tandem linked copies of the MUC1 epitope and its surrounding amino acids attached to a YFP molecule via its C-terminal (the second polypeptide).

Analysis of Cellular Expression of First and Second Polypeptides.

Cells of the mouse cell line IIC9 were transfected using standard techniques (lipofectamine transfection) with either pMUC-EYFP or pScFv-ECFP and incubated for 24 hours to allow expression of the first and second polypeptides. Protein expression by the cells was analysed using fluorescence microscopy (for pMUC-EYFP transfected cells) and immuno-blotting (Western blotting).

The results of fluorescence microscopy on cells transfected with 2 μg of pMUC-EYFP is shown in Panel A of FIG. 4. This panel shows light having a wavelength of 530 nm (corresponding to the emission spectrum of YFP) emitted from the transfected cells in response to excitation of the cells with light at 480 nm (the excitation wavelength of YFP).

The results of Western analysis (shown in FIG. 4B) demonstrated that pMUC-EYFP and pScFv-ECFP express protein products that were the predicted molecular weights for YFP labelled MUC1 and CFP labelled scFv respectively, indicating that the first and second polypeptides were being correctly expressed. The lanes shown in FIG. 4B are as follows:

1. Molecular weight markers.

2. Lysate from untransfected IIC9 cells.

3. Lysate from IIC9 cells transfected with pMUC-EYFP.

4. Lysate from IIC9 cells transfected with pScFv-ECFP.

5. Molecular weight markers.

6. Molecular weight markers.

7. Lysate from untransfected IIC9 cells.

8. Lysate from IIC9 cells transfected with pMUC-EYFP.

9. Lysate from IIC9 cells transfected with pScFv-ECFP.

10. Molecular weight markers.

Lanes 2, 3 and 4 were probed with an antibody specifically reactive to MUC1. Lanes 7, 8 and 9 were probed with an antibody that reacts equally with both CFP and YFP.

The results of the Western blotting analysis indicate that, in addition to the first and second polypeptides, the transfected cells also produce the native forms of the fluorescent proteins (indicated by arrowed bands at 27 kDa in FIG. 4B). This production of “un-linked” fluorescent proteins is most likely due to protein translation initiating at a second Kozak sequence which is present in the fluorescent protein vectors supplied by Clontech. This second Kozak sequence allows the fluorescent proteins to be expressed from the unmodified vector. Insertion of new coding sequence 5′ of the second Kozak sequence should result in only one recombinant protein being expressed from the plasmid, since the inserted material separates the Kozak sequence from the promoter. However, if the inserted sequence is small, then it is possible for the second Kozak sequence to remain too close to the mammalian CMV promotor. In this situation initiation of mRNA occurs at this second sequence as well as at the intended start codon present in the newly inserted sequence. As a result two products are produced from the vector; namely the desired recombinant protein (i.e. first or second polypeptides) and the un-linked fluorescent protein.

ScFv Coupled to Fluorescent Protein Retains its Specificity for MUC1.

In order to demonstrate that attaching a fluorescent protein does not disrupt the binding capacity of the anti-MUC1 scFv for the MUC1 epitope, IIC9 cells were transfected with pScFv-ECFP (as before), incubated for 24 hours and cell lysates prepared.

An ELISA assay was then performed using the MUC1 epitope conjugated to BSA and immobilised to the ELISA assay well and 40 μl of the respective cell lysates per ELISA well. Binding of the CFP-labelled scFv to the immobilised MUC1 epitope was confirmed with an antibody against the fluorescent protein (results shown as αMFP in FIG. 5A). A negative control for binding of the MUC1 epitope was provided by cell lysates from cells transfected with pMUC-EYFP (Neg in FIG. 5A). Binding was detected using a rabbit anti-fluorescent protein (CFP and YFP) antibody and visualised using a horseradish peroxidase (HRP) conjugated anti-rabbit secondary antibody.

Positive control for this experiment was provided by the expression product of bacteria containing the pHEN-1 vector encoding the ScFv coupled to a MYC tag. This binding was detected using a mouse anti-MYC monoclonal antibody and visualised using an HRP conjugated anti-mouse antibody.

The data shown in this figure demonstrate that the conjugation of CFP to the scFv does not disrupt the ScFv's binding to the MUC1 epitope.

MUC1 Coupled to Fluorescent Protein Remains Antigenic for ScFv.

Western analysis of lysates derived from IIC9 cells expressing the pMUC-EYFP vector has already demonstrated that the MUC epitope is being correctly expressed and is antigenic to the anti-MUC antibody in its denatured form (lane 2, FIG. 3B).

It was also important to confirm that the YFP labelled MUC1 epitope was able to bind the anti-MUC1 scFv in its native state. In order to do this, an ELISA assay was performed (using 40 μl of the respective cell lysates per ELISA well) in which bacterially derived anti-MUC1 scFv protein was immobilised to the ELISA assay well. Binding of YFP-labelled MUC1 to immobilised scFv was detected with a mouse monoclonal antibody against MUC1 (results shown as MFP in FIG. 5B) and visualised with an HRP conjugated anti-mouse antibody. Positive control was provided by a synthetic MUC1 peptide and binding detected and visualised in the same way. Negative control was provided by cell lysates from cells expressing the pScFv-ECFP vector (Neg).

In summary, these data demonstrate that attaching CFP to the anti-MUC1 scFv or YFP to the tandem repeat MUC1 epitope does not disrupt the interaction of these two proteins.

First and Second Polypeptides Interact Causing FRET in Cyto in Cells Transfected with pScFv-ECFP and pMUC-EYFP.

IIC9 cells were transfected (as before) to produce three groups of experimental cells:

1) The first group were transfected with both pScFv-ECFP and pMUC-EYFP vectors. These cells expressed YFP-labelled MUC1 epitope and a CFP-labelled anti-MUC1 intrabody.

2) The second group (negative control) were transfected with pMUC-EYFP and the unmodified ECFP-N1. These cells expressed YFP-labelled MUC1 epitope and CFP.

3) The third group (positive control) were transfected with a vector, designated pFRET, encoding a CFP linked to a YFP by the seven amino acid sequence L-Y-P-P-V-A-T.

The emission spectrum of the cells on excitation with 440 nm light was analysed using a Nikon Diphot microscope. Regions of interest were defined and a binary image applied to eliminate background. The ratio of emitted light of 530 nm wavelength to emitted light of 480 nm wavelength was then calculated. These data are illustrated in FIG. 6.

In FIGS. 6A to C pseudo-coloured images showing increase ratio of yellow light to cyan light where blue represents the lowest value, through green, yellow and red to white representing the highest ratio. Panel B illustrates cells from group 1 above, and panels A and C illustrate the positive and negative controls respectively.

Panel D is a bar graph illustrating the ratio of emitted light of 530 and 480 nm. LAD represents cells expressing the first and second polypeptides, negative the negative control group and FRET those cells from the positive control group. Bars are derived from the mean plus/minus SEM of all cells within a microscope field.

Despite the fact that both pScFv-ECFP and pMUC-EYFP produce a significant amount of unlabelled fluorescent protein, comparison of the yellow to cyan fluorescent ratio for cells expressing both scFv-CFP and MUC1-YFP against those which expressed only CFP and MUC1-YFP revealed that there was a significant increase in the fluorescent ratio when both first and second polypeptides were expressed (FIGS. 6A & B & D).

Taken in combination with the in vitro data, these in cyto data demonstrate the efficacy of intracellular analysis according to the present invention.

In order to overcome the problem of expression of unlinked fluorescent protein the inventors produced three new plasmids, pScFv-ECFP2 and pMUC-EYFP2 and pFRET2 (Sequence ID No.s 10, 11 and 12).

The sequences of these plasmids correspond to those of pScFv-ECFP, pMUC-EYFP and pFRET save that in the new plasmids the second Kozak sequence's start codon (ATG) at amino acid residues 59 (in the case of pMUC-EYFP), 260 (in the case of pScFv-ECFP) and 247 (in the case of pFRET) have been changed to ATT, using site directed mutagenesis. This change results in an amino acid change from methionine to isoleucine at the relevant residue. Cells expressing pScFv-ECFP2 and pMUC-EYFP2 vectors produce only the first and second polypeptides respectively. Cells expressing pFRET produce only the FRET control construct described above.

It will be appreciated that in the above described Example the anti-MUC1 ScFv coupled to CFP and MUC1 epitope coupled to YFP are expressed from separate vectors. An alternative embodiment of the invention is to express the anti-MUC1 ScFv linked to CFP and the MUC1 epitope linked to YFP from one expression vector. There are several ways to do this as described earlier in this document. Thus, for example, it is possible to use the commercially available dual expression vector pBudCE4.1 (Invitrogen, Paisley, UK). This vector contains two separate mammalian promoters (CMV) and (EF-1α). Using PCR and directional cloning it is possible to produce, from pMUC-EYFP and pScFv-ECFP, a vector designated herein as pBudMUC-EYFPscFvECFP, which consists of the coding sequence for the anti-MUC1 ScFv with CFP attached to its C terminus under the control of the EF-1α promotor and the coding sequence for the MUC1 epitope with YFP attached to its C terminus under the control of the CMV promoter. The complete coding sequence for this pBudMUC-EYFPscFvECFP construct is shown in Sequence ID No. 13.

References.

Griffiths and Duncan “Strategies for selection of antibodies by phage display” Current Opinion in Biotechnology 1998, 9, 102-108.

Zaccolo et al. “A genetically encoded, fluorescent indicator for cyclic AMP in living cells” Nature Cell Biology 2000, 2(1), 25-29.

1. A method of intracellularly analysing for a target molecule within a biological cell, the method comprising the steps of:
i) expressing within the cell a first polypeptide sequence comprised of a first binding species capable of binding to the target molecule and a first reporter moiety attached to the first binding species;
ii) expressing within the cell a second polypeptide sequence comprised of a second binding species capable of competing with the target molecule for binding of the first binding species and a second reporter moiety, said first and second reporter moieties being such that on binding together of the first and second binding species the first and second reporter moieties interact so as to be capable of producing a signal that can be differentiated from one capable of being generated when said first and second reporter moieties do not interact; and
iii) effecting a measurement to determine the presence or otherwise of a signal representative of binding of the first and second binding species.

2. A method as claimed in claim 1 wherein the first binding species is an antibody.

3. A method as claimed in claim 1 wherein the first binding species is an intrabody.

4. A method as claimed in claim 2 or claim 3, wherein the target molecule is a peptide.

5. A method as claimed in claim 4 wherein the second binding species is an antigen.

6. A method as claimed in claim 5 wherein the second binding species is the epitope to which the first binding species was raised.

7. A method as claimed in claim 2 or claim 3 wherein the target molecules is non-peptidic and the second binding species is an anti-antibody to the antibody or intrabody that provides the first binding species.

8. A method as claimed in any one of claims 1 to 7 wherein the first and second reporter moieties are fluorescent proteins.

9. A method as claimed in claim 8 wherein the interaction of the first and second reporter moieties is by FRET.

10. A method as claimed in claim 8 or 9 wherein one of said reporter moieties is Cyan Fluorescent Protein and the other is Yellow Fluorescent Protein.

11. A method as claimed in any one of claims 1 to 10 wherein the first and second polypeptide sequences are translated from the same RNA transcript.

12. A biological cell transfected with:
i) a first nucleic acid sequence encoding a first polypeptide sequence comprised of a first binding species capable of binding to a putative target molecule in the cell and a first reporter moiety attached to the first binding species;
ii) a second nucleic acid sequence encoding a second polypeptide sequence comprised of a second binding species capable of competing with the target molecule for binding of the first binding species and a second reporter moiety, said first and second reporter moieties being such that on binding together of the first and second binding species the first and second reporter moieties interact so as to be capable of producing a signal that can be differentiated from one capable of being generated when said first and second reporter moieties do not interact.

13. A biological cell as claimed in claim 12 wherein the first binding species is an antibody.

14. A biological cell as claimed in claim 12 wherein the first binding species is an intrabody.

15. A biological cell as claimed in claim 13 or claim 14 wherein the target molecule is a peptide.

16. A biological cell as claimed in claim 15 wherein the second binding species is an antigen.

17. A biological cell as claimed in claim 16 wherein the second binding species is the epitope to which the first binding species was raised.

18. A biological cell as claimed in claim 13 or 14 wherein the target molecules is non-peptidic and the second binding species is an anti-antibody to the antibody or intrabody that provides the first binding species.

19. A biological cell as claimed in any one of claims 12 to 18 wherein the first and second reporter moieties are fluorescent proteins.

20. A biological cell as claimed in claim 19 wherein the interaction of the first and second reporter moieties is by FRET.

21. A biological cell as claimed in claim 19 or 20 wherein one of said reporter moieties is Cyan Fluorescent Protein and the other is Yellow Fluorescent Protein.

22. A biological cell as claimed in any one of claims 12 to 21 wherein the first and second polypeptide sequences are translated from the same RNA transcript.

23. A method of producing cells according to any of claims 12 to 22, the method comprising transfecting a biological cell with:
i) a first nucleic acid sequence encoding a first polypeptide sequence comprised of a first binding species capable of binding to a putative target molecule in the cell and a first reporter moiety attached to the first binding species;
ii) a second nucleic acid sequence encoding a second polypeptide sequence comprised of a second binding species capable of competing with the target molecule for binding of the first binding species and a second reporter moiety, said first and second reporter moieties being such that on binding together of the first and second binding species the first and second reporter moieties interact so as to be capable of producing a signal that can be differentiated from one capable of being generated when said first and second reporter moieties do not interact.

24. A non-human animal comprising cells according to any of claims 12 to 22.
